(19) United States
(12) Patent Application Publication
(10) Pub. No.: US 2026/0060836 A1
(43) Pub. Date: Mar. 5, 2026

(54) Handheld Ostomy Adhesive Press Tool

(71) Applicant: Ethan Andrew Martin, Pearl River, LA (US)

(72) Inventor: Ethan Andrew Martin, Pearl River, LA (US)

(21) Appl. No.: 19/286,753

(22) Filed: Jul. 31, 2025

(51) Int. Cl. A61F 5/44; A61F 5/443

(52) U.S. Cl. CPC A61F 5/4404; A61F 5/443

(57) ABSTRACT

A handheld medical tool for improving the adhesion of ostomy pouch systems around the stoma site. The device includes a contoured pressing ring and an ergonomic handle, designed to apply uniform pressure to the adhesive surface surrounding the stoma during placement. This improves skin adhesion, reduces the risk of leakage, and enhances user confidence. The pressing ring may be manufactured in various sizes and shapes to accommodate different designs and patient anatomies, including pediatric, adult, and bariatric use cases. The tool simplifies and standardizes pouch application for patients, caregivers, and clinicians. It is also accessible for users with reduced dexterity, helping reduce inconsistent manual pressure and potential user error. Variants may include textured grips, removable rings, and convex or beveled pressing surfaces. The invention is lightweight and may be reusable or designed for single use depending on the intended setting, offering a repeatable method for achieving a reliable ostomy seal.

### BACKGROUND

Ostomy pouch systems are widely used by individuals who have undergone surgery to create a stoma for the elimination of bodily waste. These systems rely on an adhesive barrier to form a secure seal between the pouch and the patient's skin. A reliable seal is critical to prevent leakage, protect the skin from irritation, and maintain user confidence and comfort throughout daily activities.

However, achieving a consistent and uniform seal can be difficult when relying solely on manual finger pressure. The process often results in weak or uneven adhesion, leading to premature detachment, leakage, or the need for frequent pouch replacement. This challenge is not limited to patients themselves—it also affects healthcare providers, caregivers, and nurses who assist in applying ostomy appliances, particularly in clinical or long-term care settings where time and consistency are critical.

The inventor became aware of these challenges through a family member who uses an ostomy pouch system and had frequent issues with the stoma seal ring prematurely failing. Observing that nurses and caregivers also struggled to apply even pressure during installation, the inventor recognized the need for a tool that could improve the sealing process. This led to the development of a simple, hand-held pressing tool designed specifically to apply uniform pressure across the adhesive surface. The tool enhances the bond between the pouch and the skin barrier, reducing failure rates and improving wear time, while also simplifying the application process for both users and caregivers.

### SUMMARY

The present invention is a handheld tool designed to improve the sealing process of ostomy pouch systems by applying consistent and uniform pressure to the adhesive surface surrounding the stoma during installation. The device consists of a rigid or semi-flexible body with a shaped pressing ring, contoured to target the adhesive surface directly surrounding the stoma. It is available in various sizes and geometries to accommodate different stoma dimensions, patient anatomies, and ostomy appliance designs. An ergonomic handle or grip allows the user—whether a patient or caregiver—to comfortably apply downward force, ensuring an even seal between the adhesive ring and the user's skin.

Unlike conventional methods that rely on inconsistent hand pressure or ad-hoc pressing techniques, this tool standardizes the sealing process, reducing the likelihood of leaks, improving adhesive performance, and extending wear time. The tool may be manufactured in a range of shapes (e.g., circular, oval, convex, or contoured) and sizes to accommodate various adhesive barrier configurations and user anatomies, including pediatric, adult, and bariatric cases. It may be constructed from medical-grade plastic, rubber, or a combination of materials optimized for comfort, sterilization, and/or repeat use, depending on the intended setting or user.

This invention simplifies the pouch application process, reduces seal failures, and provides a reliable, repeatable method of achieving proper adhesion for both home users and medical professionals. It is intended to cover multiple embodiments that share the same core function: the application of uniform pressure to an ostomy adhesive barrier.

### DRAWING DESCRIPTION

FIG. 1 is a side view of the handheld ostomy adhesive press tool (11), illustrating the vertical profile of the device, including the ergonomic handle (12) and pressing ring (14). Hidden lines show the internal cavity (13) cut into the body of the tool to a depth of approximately 40 mm, providing clearance for the user's stoma.

FIG. 2 is a top-down plan view of the tool, showing the overall circular geometry of the pressing surface and the general footprint of the ergonomic handle (12).

FIG. 3 is a bottom view of the device, highlighting an oval-shaped inner cavity (13). This cavity is open upwardly to allow placement over the user's stoma. The outer lip (14), also referred to as the pressing ring, is the portion of the tool that engages the adhesive zone during use.

FIG. 4 is a bottom view of a circular-shaped variant of the device, featuring an inner cavity (13). Like FIG. 3, this configuration demonstrates the tool's adaptability to variations in stoma shape and size, ensuring proper alignment and consistent adhesive contact. Although the stoma (18) and adhesive zone (17) are not shown in this view, they are illustrated in FIGS. 8-10 to demonstrate how this configuration achieves uniform pressure around the stoma perimeter, improving seal performance and reducing the risk of leakage.

FIG. 5 is an isometric view of the tool (11) from a side angle, offering a three-dimensional representation of the overall form and proportions.

FIG. 6 is a bottom-side isometric view of the tool (11), showing the oval-shaped cavity (13) and beveled pressing ring (14). The figure illustrates the curvature and open cavity depth that enable the device to conform closely to a user's stoma region. Although the stoma (18) and adhesive zone (17) are not shown in this view, they are illustrated in FIGS. 8-10 to demonstrate how this configuration achieves uniform pressure around the stoma perimeter, improving adhesive performance and minimizing the risk of leakage.

FIG. 7 is a bottom-side isometric view of the circular variant of the tool (11). It shows the same structural features as FIG. 6 but with a circular cavity (13) instead of an oval one. This figure demonstrates the tool's adaptability for different stoma sizes and adhesive barrier geometries. Although the stoma (18) and adhesive zone (17) are not shown in this view, they are illustrated in FIGS. 8-10 to demonstrate how this configuration achieves uniform pressure around the stoma perimeter, improving adhesive performance and minimizing the risk of leakage.

FIG. 8 illustrates the tool in use, with a simplified hand (15) pressing the tool (11) downward onto an ostomy pouch (16). The pouch is shown in its natural configuration during application, with the adhesive backing positioned downward and the adhesive zone (17) facing upward toward the tool. This orientation represents how the user applies pressure through the tool to activate adhesion between the pouch's underside and the user's skin, ensuring consistent contact and seal integrity.

FIG. 9 depicts the tool in use on a prone model (19). The user is lying on their back with the ostomy pouch (16) adhered to the abdomen, and the press tool (11) is applied through the non-adhesive outer surface of the pouch to reinforce adhesion between the pouch's backing and the skin. The hand (15) maintains a steady downward pressure over the adhesive zone (17) to ensure even contact and seal security while the user is in a reclined position.

FIG. 10 illustrates a standing model (19) being assisted by another user who is holding the press tool (11) in a poised position above the ostomy pouch (16). The pouch is shown adhered to the user's abdomen with the adhesive zone (17) surrounding the stoma (18). This figure depicts a preparatory action prior to pressing, demonstrating how the tool can be positioned and operated by a caregiver to apply uniform pressure over the pouch's non-adhesive outer surface, reinforcing adhesion around the stoma while maintaining portability and ease of use.

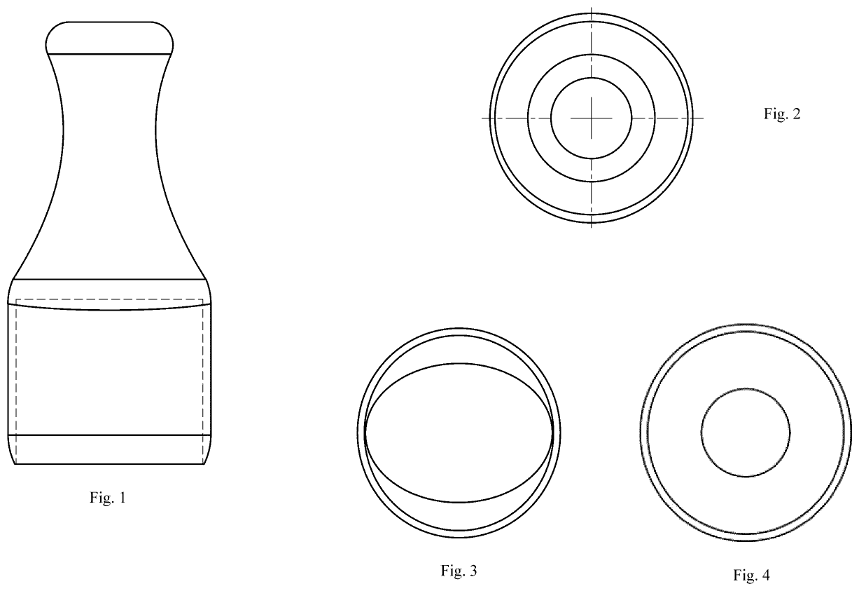

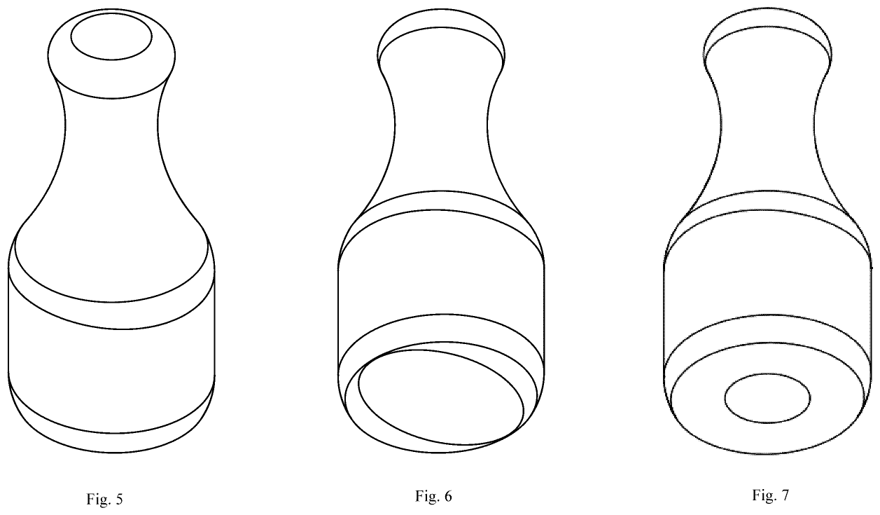

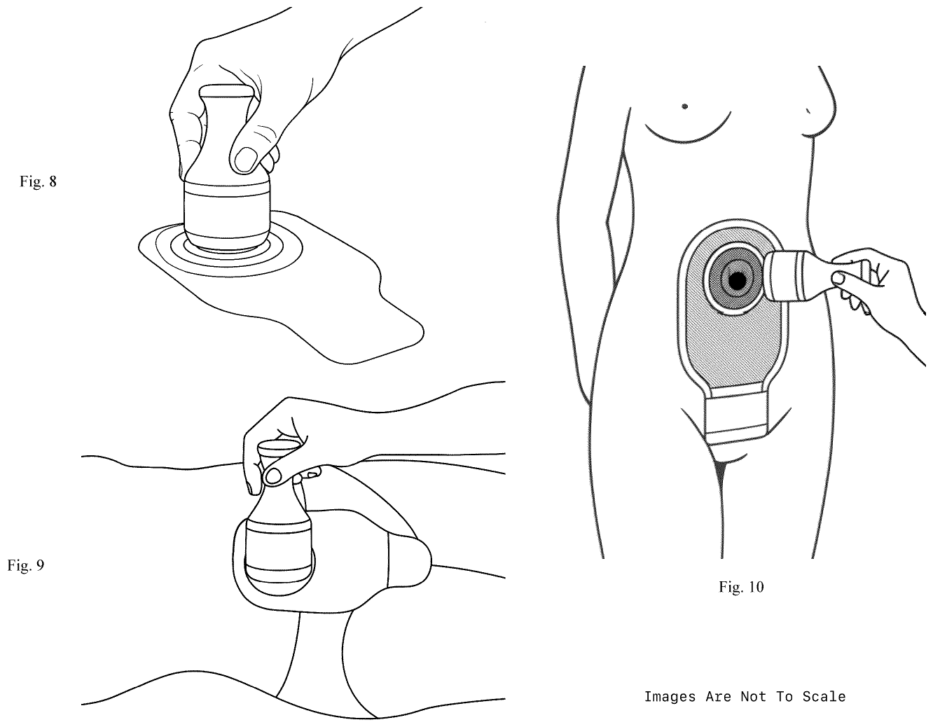

### DETAILED DESCRIPTION

The handheld ostomy adhesive press tool is a one-piece device composed of a contoured pressing ring and an ergonomic handle. It is designed to apply uniform, controlled pressure across the adhesive surface surrounding the stoma to improve seal integrity during application.

### Use Case/Installation Process

In operation, the user first prepares a pre-cut ostomy pouch system, aligning the opening of the adhesive barrier to match the stoma site. Once the backing is removed and the pouch is placed over the stoma, the user positions the handheld ostomy press tool so that the pressing ring surrounds the stoma. Gentle downward pressure is then applied using the ergonomic handle, ensuring uniform force is distributed through the beveled pressing ring. This ensures optimal adhesive contact with the peristomal skin, reducing the likelihood of leakage or edge lifting. The process can be performed by the patient or a caregiver and may be repeated to ensure complete contact across all adhesive zones.

### Pressing Ring

The tool's base features a beveled pressing ring with an outer width of approximately 49.1 mm. The ring's pressing edge has a curved profile radius of approximately 15.4 mm, optimized to follow the contour of standard adhesive barriers. The beveled contact surface that engages with the ostomy adhesive barrier is approximately 45.2 mm wide, allowing for broad, even distribution of force without concentrated pressure points. This shape minimizes user fatigue and avoids damage to the skin or adhesive.

The pressing ring may be produced in a variety of shapes and sizes to closely follow the perimeter of the stoma, including but not limited to circular, oval, elliptical, irregular, or custom geometries. These variations are intended to accommodate patient-specific anatomy, stoma geometry, and differing adhesive barrier configurations, such as those used in pediatric, adult, or bariatric settings. Such modifications may be made without departing from the spirit or scope of the invention.

All stated dimensions are provided for illustrative purposes only and may be adjusted to accommodate individual patient needs or specific ostomy pouch configurations without departing from the scope of the invention.

### Handle Design

Integrated into the top of the press ring is a contoured vertical handle that extends the total tool height to approximately 102.6 mm. The handle is shaped for comfortable grip, allowing both patients and caregivers to apply downward pressure with stability and control. Its broad top surface provides ample area for the palm or fingers and may be textured or over molded with a soft elastomer for improved comfort and slip resistance.

### Material and Manufacturing

The tool may be constructed from any suitable flexible or semi-rigid, medical-grade plastic, allowing for adequate structural integrity while maintaining user comfort. Materials may be selected based on the intended use case, including considerations for skin safety, disinfect ability, and durability. The device may be manufactured through injection molding for high-volume production or 3D printing for prototyping or limited runs. Textured or elastomeric surface treatments may be applied to the handle or pressing ring to improve grip and user interface. The overall design is lightweight and features a smooth surface finish to facilitate cleaning, disinfection, or disposal depending on use context.

### Alternative Embodiments

The handheld ostomy adhesive press tool may be offered in multiple sizes, ranging from small (for pediatric use) to extra-large (for bariatric applications), to accommodate various patient anatomies and stoma configurations. The pressing ring may be circular, oval, convex, or shaped according to specific adhesive barrier profiles. Additionally, modular designs may include interchangeable pressing heads or adjustable handle lengths for use in clinical, home, or travel settings. The device may also include color-coded versions for easy identification of size or type in clinical environments.

All such variations are considered within the scope of the present invention, provided they maintain the essential function of delivering uniform adhesive pressure to the adhesive surface surrounding the stoma. In some embodiments, the device may be manufactured as a single-use sterile unit using cost-effective materials suitable for clinical distribution, or as a multi-use, disinfectant-compatible version intended for repeated personal or caregiver use.

1. A handheld medical tool for ostomy care comprising:
a pressing ring having a top surface and a bottom surface;
an ergonomic handle integrally formed with or affixed to the top surface of the pressing ring;
wherein the pressing ring defines a central opening and is configured to surround a stoma without contacting it; and
wherein the bottom surface of the pressing ring includes a beveled or curved contact surface for applying uniform, downward pressure to secure an adhesive barrier to the skin.

2. A method for securing an ostomy pouch to a user comprising:
providing a handheld tool with a contoured pressing ring and ergonomic handle;
positioning the pressing ring over the adhesive barrier of the ostomy pouch surrounding the stoma; and
manually pressing downward to apply even pressure to the adhesive region without contacting the stoma.

3. A system for ostomy pouch application comprising:
an ostomy adhesive press tool having a hollow central cavity for stoma clearance;
a pressing ring configured to match the adhesive footprint surrounding the stoma; and
an integrated handle shaped to allow single-handed application of downward force for secure adhesion.

4. The tool of claim 1, wherein the pressing ring is formed of a flexible or semi-rigid medical-grade material selected from the group consisting of plastics, elastomers, or other biocompatible polymers suitable for skin contact and repeatable or disposable use.

5. The tool of claim 1, wherein the pressing ring has a contact width of between 10 mm and 100 mm.

6. The tool of claim 1, wherein the ergonomic handle is contoured for a palm grip and optionally includes a textured or over-molded surface for improved comfort and slip resistance.

7. The tool of claim 1, wherein the central opening is generally oval, circular, or custom-shaped and is hollow upward from the bottom surface to accommodate the protrusion of the stoma during use.

8. The tool of claim 1, wherein the pressing ring includes a beveled or rounded edge configured to distribute pressure evenly without damaging the adhesive barrier or skin.

9. The tool of claim 1, wherein the handle and pressing ring are formed as a single integrated unit or as modular components assembled together.

10. The tool of claim 1, wherein the device is intended for single use or multiple use following disinfection or sterilization, in either clinical or home care settings.

11. The tool of claim 1, wherein the tool is operable by a patient, caregiver, or healthcare professional using a single hand.

12. The tool of claim 1, wherein the device is manufactured using injection molding, 3D printing, or another suitable method for medical device fabrication.

13. The tool of claim 1, wherein the handle includes a textured, contoured, or elastomeric grip surface to enhance comfort and control during operation.

14. The tool of claim 1, wherein the pressing ring is provided in multiple size variations corresponding to different stoma dimensions or patient anatomies.

15. The tool of claim 1, wherein the geometry of the pressing ring is adaptable to pediatric, adult, or bariatric users.

16. The tool of claim 1, wherein the device is optionally marked or color-coded to distinguish size, shape, or intended user group.

17. The tool of claim 1, wherein the handle is angled relative to the pressing ring to improve ergonomic performance.

18. The tool of claim 1, wherein at least a portion of the pressing ring is transparent or translucent to allow visual alignment of the adhesive barrier with the stoma.

19. The tool of claim 1, wherein a removable, single-use hygienic cover or cap is fitted to the pressing ring for sterile application.

20. The tool of claim 1, wherein the handle includes a flared or winged section near its base to prevent hand slippage and increase stability during use.
